(19) United States
(12) Patent Application Publication
(10) Pub. No.: US 2024/0016191 A1
(43) Pub. Date: Jan. 18, 2024

(54) METHOD FOR PREPARING AGROCYBE CYLINDRACEA FERMENTED KUDZUVINE ROOT AND COIX SEEDS BEVERAGE

(71) Applicant: Guizhou University, Guiyang (CN)

(72) Inventors: Laping HE, Guiyang (CN); Cuiqin LI, Guiyang (CN); Yun YANG, Guiyang (CN)

(21) Appl. No.: 17/982,611

(22) Filed: Nov. 8, 2022

(30) Foreign Application Priority Data
Jul. 15, 2022 (CN) 202210836031.5

(51) Int. Cl. A23L 2/38; A61K 36/07; A61K 36/488; A61K 36/8994; A23L 7/25

(52) U.S. Cl. CPC A23L 2/382; A61K 36/07; A61K 36/488; A61K 36/8994; A23L 7/25; A61K 2236/15; A61K 2236/19; A23V 2002/00

(57) ABSTRACT

Disclosed is a method for preparing Agrocybe cylindracea fermented kudzuvine root and coix seeds beverage, and relates to the technical field of fermented beverage product development. The method includes steps of germinating coix seeds to obtain germinated coix seeds, respectively pulping kudzuvine root and germinated coix seeds with water into pastes of kudzuvine root and germinated coix seeds, gelatinizing the pastes, followed by respectively adding with high-temperature amylase for primary enzymolysis and inactivating enzyme, and respectively adding with glucoamylase for secondary enzymolysis and inactivating enzyme to obtain saccharified liquids of kudzuvine root and germinated coix seeds; mixing the saccharified liquids of kudzuvine root and germinated coix seeds, followed by sterilizing and inoculating with Agrocybe cylindracea seed solution for fermentation, and then obtaining a Agrocybe cylindracea fermented kudzuvine root and coix seeds beverage.

### CROSS-REFERENCE TO RELATED APPLICATIONS

This application claims priority to Chinese Patent Application No. 202210836031.5, filed on Jul. 15, 2022, the contents of which are hereby incorporated by reference.

### TECHNICAL FIELD

The present application relates to the technical field of developing fermented beverage products, and particularly a method for preparing Agrocybe cylindracea fermented kudzuvine root and coix seeds beverage.

### BACKGROUND

Nowadays people' preferences and demands for some products in daily life, such as beverages, have changed significantly due to the general improvement of the living standard, as people require not only good organoleptic properties but also nutritional functions, leading to a significant increase in sales of beverages with bioactivity and nutritional health functions in recent years. Fermented beverage, a product produced by the action of microorganisms or enzymes in order to produce the desired biochemical changes, is therefore becoming ever more popular around the world due to its high nutritional value and good microbial stability. The microorganisms most commonly used in preparing fermented beverages include yeasts, lactic acid bacteria, bifidobacteria, and lower fungi. Currently, beverages produced by fermentation using edible fungi are rarely seen on the market.

### SUMMARY

Based on the above content, the present application provides a method for preparing Agrocybe cylindracea fermented kudzuvine root (Radix Pueraria lobata) and coix seeds (Coix Lacryma-jobi L.) beverage, and the prepared fermented beverage has good flavor, health care functions and sensory quality.

To achieve the above objectives, the present application provides the following technical schemes:
one of the technical schemes of the present application is a method for preparing Agrocybe cylindracea fermented kudzuvine root and coix seeds beverage, including:
step 1, germinating coix seeds to obtain germinated coix seeds;
step 2, pulping kudzuvine root with water into a paste of kudzuvine root, gelatinizing the paste of kudzuvine root, followed by adding high-temperature amylase for primary enzymolysis and inactivating enzyme, and adding glucoamylase for secondary enzymolysis and inactivating enzyme to obtain saccharified liquid of kudzuvine root; pulping germinated coix seeds with water to get a paste of germinated coix seeds, gelatinizing the paste of germinated coix seeds, followed by adding with high-temperature amylase for primary enzymolysis and inactivating enzyme, and adding with glucoamylase for secondary enzymolysis and inactivating enzyme to obtain saccharified liquid of germinated coix seeds; and
step 3, mixing the saccharified liquids of kudzuvine root and germinated coix seeds, then sterilizing and inoculating with Agrocybe cylindracea seed solution for fermentation, then obtaining a fermented beverage prepared using kudzuvine root, coix seeds and agrocybe cylindracea.

Optionally, the germinated coix seeds is step 1 are obtained by: subjecting coix seeds to germinating treatment in an environment with a temperature of 27-31 degree Celsius (° C.) and a humidity of 88-93 percent (%) for 22-28 hours (h); rather optionally, the coix seeds are germinated at 29° C. with a humidity of 90% for 24 h.

Optionally, the coix seeds are disinfected before subjecting to germinating treatment, including: soaking coix seeds in 1% sodium hypochlorite solution for 15 minutes (min) for disinfection treatment, and washing the coix seeds with deionized water until there is no smell of sodium hypochlorite; soaking the sterilized coix seeds in the water of 10 times a volume at 36° C. for 10 h to fully absorb water; and soaking the coix seeds in 1% sodium hypochlorite solution for 15 min, followed by washing with deionized water until there is no smell of sodium hypochlorite.

Optionally, as preparing the saccharified liquid of kudzuvine root in step 2, the kudzuvine root is in a mass-volume ratio of 1 gram (g): 8-10 milliliters (mL) with water during a process of pulping into a paste; optionally, the kudzuvine root is in a mass-volume ratio of 1 g: 10 mL; the gelatinizing refers to gelatinizing at 85-95° C. for 25-35 min, and preferably includes gelatinizing at 90° C. for 30 min; the high-temperature amylase added in step 2 is in a concentration of 180-220 micrograms (u/g), and preferably 200 u/g; optionally, the primary enzymolysis specifically includes heating at 85-95° C. for 40-50 min, preferably heating at 90° C. for 45 min;
as preparing the saccharified liquid of germinated coix seeds in step 2, the saccharified liquid of germinated coix seeds is in a mass-volume ratio of 1 g: 8-10 mL with water during a process of pulping into a paste; the gelatinizing refers to gelatinizing at 85-95° C. for 25-35 min, and preferably includes gelatinizing at 90° C. for 30 min; the high-temperature amylase added is in a concentration of 180-220 u/g, and preferably 200 u/g; and the primary enzymolysis specifically includes heating at 85-95° C. for 40-50 min, preferably heating at 90° C. for 45 min.

Optionally, as preparing the saccharified liquids of kudzuvine root and germinated coix seeds in step 2, the glucoamylase is added in a concentration of 280-320 u/g, preferably 300 u/g; and the secondary enzymolysis includes heating at 60-70° C. for 75-85 min, and preferably at 65° C. for 80 min.

Optionally, the saccharified liquids of kudzuvine root and germinated coix seeds are in a volume ratio of 3: 7-7:3; preferably, the volume ratio is 1:1.

Optionally, the sterilizing in step 3 is carried out at 118-125° C. for 18-25 min; preferably, the sterilizing is carried out at 121° C. for 20 min.

Optionally, the Agrocybe cylindracea seed solution is inoculated with an amount of 4-6 weight percentage (wt %), preferably 5 wt %; the fermentation is carried out under a rotating speed of 150-190 rotations per minute (r/min) at 25-29° C. for a duration of 2-4 days, rather optionally, the fermentation is carried out under a rotating speed of 165 r/min at 26.5° C. for 3.5 days; optionally, the fermentation also includes a step of adding auxiliary materials for conditioning, including: adding 2 wt % white sugar, 1.5 wt % non-dairy creamer, 0.05 wt % xanthan gum and 0.04 wt % pectin for conditioning.

Optionally, the Agrocybe cylindracea seed solution is prepared as follows:
(1) slant culture of original seed: selecting mycelium block from a master test tube, inoculating the mycelium block to slant culture medium, and culturing the medium under constant temperature until thallus grows all over the slant; and
(2) liquid seed culture: selecting mycelium block from the cultivated slant, inoculating the mycelium block into liquid seed culture medium, and homogenizing after culturing at a shaking table under constant temperature; inoculating the homogenized seed solution into the liquid seed culture medium in an inoculating amount of 3.5-4.5 wt %; and culturing at a shaking table under constant temperature to obtain the Agrocybe cylindracea seed solution.

Another objective of the present application is to provide an Agrocybe cylindracea fermented kudzuvine root and coix seeds beverage.

### Technical Conception of the Present Application

edible fungi are important biological resources with rich proteins, carbohydrates, vitamins and mineral elements, in addition to rich extracellular and intracellular enzymes; moreover, cellulase, hemicellulase and ligninase produced by edible mushrooms can decompose cellulose, hemicellulose and lignin that are not available to plants and animals in general; during submerged fermentation of edible fungi, some ingredients in raw materials are degraded by submerged fermentation of edible fungi and many new nutritional ingredients and flavor substances are produced accordingly, making edible fungi that produce natural nutritional and flavor substances an ideal choice for developing new fermentation systems so as to meet consumers' demand for natural foods; with such properties that other fermented beverages cannot be compared and replaced, edible fungi fermented beverages embrace a broad potential for development and application;

coix seed (Coix lacryma-jobi L.), also known as the seed of Job's tears, pearl barley, and barley kernels, is one of the common health-promoting cereal crops and is regarded as the “king of grains”; the coix seed contains all the amino acids in a ratio that is very close to the needs of human body, and more protein than rice and more fat than most grains; apart from being rich in carbohydrates, fats, proteins, essential amino acids, etc., coix seed also contains relatively unique components including coixenol, coixol (also known as coixenolide) and coix polysaccharide; along with the increasing awareness of consumers for health care, coupled with the medicinal and food properties, there has been an increasing and extensive research and development of coix seeds related products; according to the present application, coix seeds are subjected to germination, a low-cost process to improve the utilizing rate of common grain varieties and diversify grains, so as to give full play to the nutritional value of coix seeds and improve the nutritional quality of chemical components in the target plant;

kudzuvine root (Radix Pueraria lobata), also known as yellow kudzu and pachyrhizua angulatus, is the dried root of deciduous vine Pueraria lobota, a perennial of the legume family; kudzuvine root is also renowned as “thousand-year ginseng” for containing a large number of ingredients beneficial to human body, including starch, dietary fiber, isoflavones, polysaccharides, various essential amino acids and terpenoids required by human growth and development, etc.; such a high-quality plant for both medicine and food has been mainly developed into powerful drugs utilizing its active ingredients as a result of increasing awareness of health care; yet, there is no record of preparing functional foods from kudzuvine root via fermentation; therefore, it is important to fully utilize the resources of kudzuvine root by expanding the development and application of kudzuvine root in order to provide excellent health products and therefore improve people's health.

The present application discloses the following technical effects:
the method for preparing Agrocybe cylindracea fermented kudzuvine root and coix seeds beverage provided by the present application uses coix seeds with health functions as grain raw material, where the coix seeds are germinated to have improved flavor and increased nutrition, and then the germinated coix seeds are compounded with plant material kudzuvine root, followed by fermentation by Agrocybe cylindracea, resulting in a compound fermentation broth of kudzuvine root and coix seeds with improved biological activity, in addition to a polysaccharide content of 8.41 milligrams per milliliter (mg/mL) and γ-aminobutyric acid (GABA) content of 4.2 mg/100 mL; meanwhile, the compound fermentation broth is of uniform brown color, with both the rich flavor of Agrocybe cylindracea and the fragrance of coix seedcoix seeds, and with no offensive odor; the sensory properties are also effectively improved through fermentation, resulting in a high quality beverage with good flavor, health care function and sensory properties.

According to the method of the present application, kudzuvine root, coix seeds and Agrocybe cylindracea are reasonably utilized and converted into high value-added products from common agricultural products; economic value is created by optimizing and upgrading the industrial chain configuration of kudzuvine root, coix seeds and Agrocybe cylindracea resources, and the planting industries of kudzuvine root and coix seeds are simultaneously developed, which are beneficial to the improvement of natural ecology. The fermented beverage of kudzuvine root and coix seed developed by the present application fills the blank of similar products in the market.

### BRIEF DESCRIPTION OF THE DRAWINGS

For a clear illustration of the technical schemes in the embodiments or prior art of the present application, the drawings to be used in the embodiments are briefly described below, and it is evident that the drawings in the following description are only some embodiments of the present application, and that other drawings may be obtained from these drawings without creative work for a person of ordinary skill in the art.

FIG. 1 is a chart showing total ion flow of volatile flavor components before fermentation in Embodiment 1.

FIG. 2 is a chart showing total ion flow of volatile flavor components in the fermentation of Embodiment 1.

FIG. 3 is a chart showing total ion flow of volatile flavor components after fermentation in Embodiment 1.

FIG. 4 is a chart showing total ion flow of volatile flavor components in a beverage prepared in Embodiment 1.

FIG. 5A-FIG. 5C show influence of different inoculation amount on sensory score, puerarin, coixol, γ-aminobutyric acid (GABA) and polysaccharide content of agrocybe cylindracea fermented kudzuvine root and coix seeds beverage in Embodiment 4, where FIG. 5A is a sensory radar chart, FIG. 5B shows the influence of inoculation amount on contents of puerarin and coixol, and FIG. 5C shows the influence of inoculation amount on contents of GABA, crude polysaccharide, and sensory score.

FIGS. 6A-6C show influence of different rotating speeds on sensory score, puerarin, coixol, GABA and polysaccharide content of Agrocybe cylindracea fermented kudzuvine root and coix seeds beverage in Embodiment 4, where FIG. 6A is the sensory radar chart, FIG. 6B shows the influence of rotating speed on puerarin and coixol, and FIG. 6C shows the influence of rotating speed on contents of GABA, crude polysaccharide, and sensory score.

FIGS. 7A-7C show influence of different fermentation temperatures on sensory score, puerarin, coixol, GABA and polysaccharide content of kudzuvine root and coix seeds beverage fermented by Agrocybe cylindracea according to Embodiment 4, where FIG. 7A is the sensory radar chart, FIG. 7B shows the influence of fermentation temperature on puerarin and coixol, and FIG. 7C shows the influence of fermentation temperature on contents of GABA, crude polysaccharide, and sensory score.

FIGS. 8A-8C show influence of different fermentation duration on sensory score, puerarin, coixol, GABA and polysaccharide content of kudzuvine root and coix seeds beverage fermented by Agrocybe cylindracea in of Embodiment 4; where FIG. 8A is a sensory radar chart, FIG. 8B shows the influence of fermentation duration on puerarin and coixol, and FIG. 8C shows the influence of fermentation duration on GABA, crude polysaccharide, and sensory score.

FIG. 9 shows a processing of preparing the Agrocybe cylindracea fermented kudzuvine root and coix seeds beverage according to one of the embodiments of the present application.

### DETAILED DESCRIPTION OF THE EMBODIMENTS

Now various exemplary embodiments of the present application will be described in detail. This detailed description should not be taken as a limitation of the present application, but should be understood as a more detailed description of some aspects, characteristics and embodiments of the present application.

It should be understood that the terms mentioned in the present application are only used to describe specific embodiments, and are not used to limit the present application. In addition, for the numerical range in the present application, it should be understood that each intermediate value between the upper limit and the lower limit of the range is also specifically disclosed. Every smaller range between any stated value or the intermediate value within the stated range and any other stated value or the intermediate value within the stated range is also included in the present application. The upper and lower limits of these smaller ranges may be independently included or excluded from the range.

Unless otherwise stated, all technical and scientific terms used herein have the same meanings commonly understood by those of ordinary skill in the field to which this application relates. Although the present application only describes preferred methods and materials, any methods and materials similar or equivalent to those described herein can be used in the practice or testing of the present application. All documents mentioned in this specification are incorporated by reference to disclose and describe the methods and/or materials related to the documents. In case of conflict with any incorporated documents, the contents of this specification shall prevail.

Without departing from the scope or spirit of the present invention, it is obvious to those skilled in the art that many modifications and changes may be made to the specific embodiments of the present specification. Other embodiments obtained from the description of the present invention will be obvious to the skilled person. The description and embodiment of that invention are only exemplary.

As used in this paper, the terms “comprising”, “including”, “having” and “containing” are all open terms, meaning including but not limited to.

In the present application, “percent (%)” refers to mass percentage unless otherwise specified.

The embodiments of the present application utilize a kind of edible fungi (poria cocos, agrocybe cylindracea, Grifola frondosa, lentinus edodes and Pleurotus ostreatus) seed solution, and the seed solution is prepared as follows:
(1) slant culture of original seed: selecting mycelium block of suitable size from master test tube, inoculating the mycelium block to slant culture medium using an inoculation spatula, and culturing the medium under constant temperature of 27 degree Celsius (° C.) until thallus grows all over the slant; and
(2) liquid seed culture: selecting mycelium block of suitable size from the cultivated slant, inoculating the mycelium block into liquid seed culture medium (250 milliliters (mL) triangular bottle with 100 mL liquid volume), placing the medium in a constant temperature shaker at 27° C. and 170 rotations per minute (r/min) for incubation of 7 days; pouring the seed solution after incubation into a sterilized beaker in clean bench and homogenizing for 30 seconds (s); inoculating the homogenized seed solution into the liquid seed culture medium in an inoculating amount of 4 wt %; and culturing at a shaking table under constant temperature of 27° C. and 170 r/min for 12 hours (h) to obtain the edible fungi seed solution.

The embodiments of the present application use edible fungus of Agrocybe cylindracea F4 provided by Edible Fungus Research Institute of Xishui County, Guizhou Province.

The raw materials used in the embodiments of the present application are purchased unless otherwise specified.

### Embodiment 1

S1, selecting undamaged coix seeds, soaking coix seeds in 1% sodium hypochlorite solution for 15 min for disinfection, and washing the coix seeds after soaking with deionized water until there is no smell of sodium hypochlorite; soaking the sterilized coix seeds in 10 times a volume of 36° C. water for 10 h to fully absorb water, followed by soaking in 1% sodium hypochlorite solution for 15 min and washing with deionized water until there is no smell of sodium hypochlorite; spreading the coix seeds between two layers of gauze (the gauze is sterilized at 121° C. for 20 min), and placing the gauze in an incubator with a temperature of 29° C. and a humidity of 90% for germination for 24 h (with water sprayed every 12 h during the germination process to keep it moist), obtaining germinated coix seeds for later use;
S2, pulping kudzuvine root powder with water at a ratio of 1:10 (weight/volume, w/v), gelatinizing at 90° C. for 0.5 h, adding high-temperature resistant amylase at a concentration of 200 micrograms (u/g), water-bathing at 90° C. for 45 min, and inactivating enzyme at 100° C. for 10 min; then adding glucoamylase at a concentration of 300 u/g, water-bathing at 65° C. for 80 min, and taking out and inactivating enzyme for 10 min, naturally cooling to room temperature to obtain saccharified liquid of kudzuvine root for later use;
pulping the germinated coix seeds prepared in step (1) with water at a ratio of 1:8 (w/v), gelatinizing at 90° C. for 0.5 h, adding high-temperature resistant amylase at a concentration of 200 u/g, water-bathing at 90° C. for 45 min, and inactivating enzyme at 100° C. for 10 min; then adding glucoamylase at a concentration of 300 u/g, water-bathing at 65° C. for 80 min, and taking out and inactivating enzyme for 10 min, naturally cooling to room temperature to obtain saccharified liquid of germinated coix seeds;
S3, mixing the above-mentioned saccharified liquids of kudzuvine root and germinated coix seeds according to a volume ratio of 1:1 to obtain a compound liquid;
S4, sterilizing the compound liquid at 121° C. for 20 min;
S5, inoculating Agrocybe cylindracea seed solution into the sterilized compound liquid with an inoculation amount of 5%, and carrying out constant temperature fermentation in a shaking table with a rotation speed of 165 r/min, fermentation temperature of 26.5° C. and fermentation duration of 3.5 days, obtaining a fermentation broth; and
S6, following the fermentation, adding 2% sugar of a total mass of the fermentation broth, 1.5% phytolipid, 0.05% xanthan gum and 0.04% pectin for conditioning to prepare a beverage.

The beverage is tested and analyzed in terms of components before and after fermentation in the present embodiment.

Headspace solid-phase microextraction is used to analyze volatile flavor substances in beverages (samples), with specific operations as follows:
sample treatment: putting 5.0 mL sample in a 20 mL top empty bottle, adding with 10 microliters (μL) 2-octanol with a concentration of 5 mg/L as an internal standard, and sealing for later use;
extraction conditions: extraction temperature of 50° C., extraction duration of 30 min, and desorption duration of 3 min;
GC-MS conditions: Pegasus HRT 4D Plus full two-dimensional gas chromatography-high-throughput high-resolution mass spectrometry is used to analyze the solid-phase microextraction components of beverages before and after fermentation and finished products. A DB-Wax (30 meters (m)*0.25 millimeter (mm)*0.25 micrometer (um)) capillary column is used, with an initial column temperature of 40° C., maintained for 3 min, and then increased to 230° C. at 10° C./min and maintained for 6 min. Helium (1 mL/min) is used as a carrier gas, and the temperature of injector and detector is 250° C.; electron impact (EI) ion source, with electron energy of 70 electronvolts (eV), the temperature of ion source is set to be 200° C. and the temperature of interface is kept at 250° C.

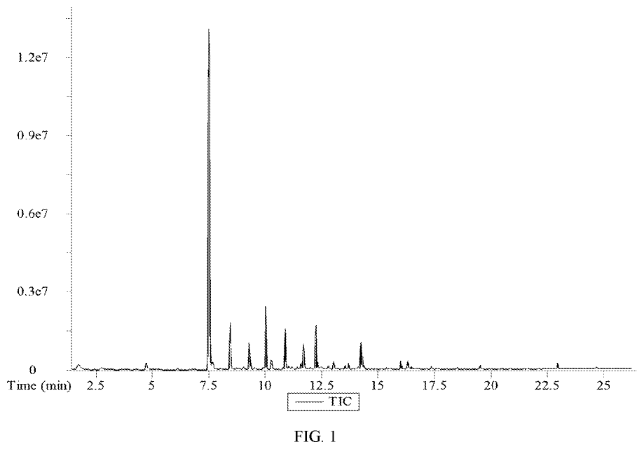

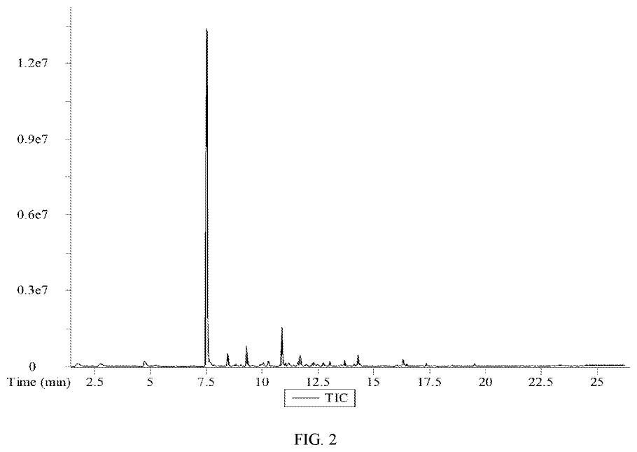

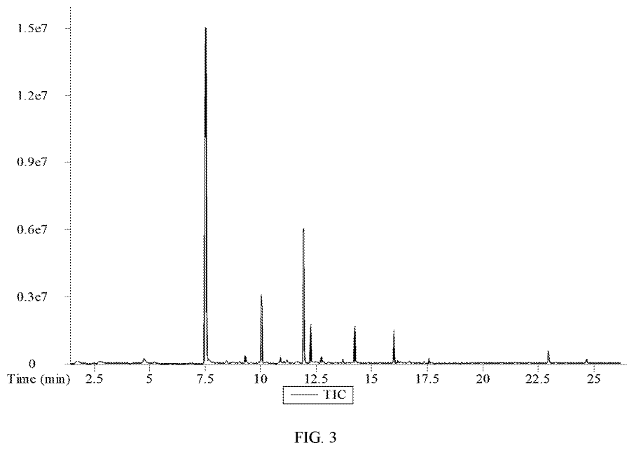

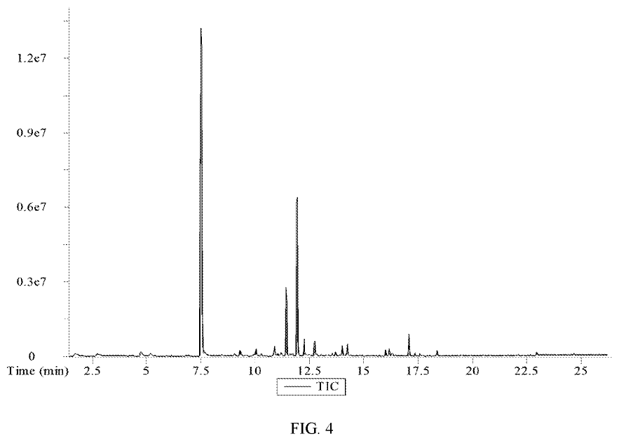

Volatile compounds are identified by comparing their mass spectra with those of standards from the MS library of National Institute of Standards and Technology (NIST2020.L). By comparing the retention indices and mass spectra of the components, the substances are characterized and the content of each volatile substance in the sample is calculated by the internal standard method. See Table 1 for the types and total amount of volatile flavor substances in the beverage, and see FIG. 1, FIG. 2, FIG. 3 and FIG. 4 for total ion flow charts of volatile compounds in the beverage.

TABLE 1

| | Absolute content (microgram (μg)/100 mL) | | | |
|---|---|---|---|---|
| | Pre- | Fermentation | After | Finished |
| | fermentation | for 42 h | fermentation | product |
| Aldehydes | 17.675 | 14.697 | 8.346 | 10.287 |
| Ketone | 13.484 | 7.102 | 3.883 | 2.817 |
| Esters | 6.843 | 0.800 | 5.398 | 22.146 |
| Alcohol | 15.648 | 13.468 | 3.206 | 1.331 |
| Acids | 5.736 | 4.951 | 0.834 | 1.852 |
| Hydrocarbon | 2.757 | 0.642 | 1.196 | 1.417 |
| Phenols | 0.163 | 0.671 | 0.216 | 3.949 |
| Aromatic | 0.440 | 2.353 | 1.478 | 5.326 |
| Furan | 14.318 | 4.717 | 2.345 | 1.101 |
| compound | | | | |
| Pyrazine | 3.060 | 3.182 | 3.108 | 2.674 |
| compound | | | | |
| Others | 2.319 | 2.140 | 1.917 | 1.733 |

It is seen from Table 1 and FIGS. 1-4 that the types and quantities of volatile compounds have changed after fermentation. Generally speaking, with the progress of fermentation, the contents of aldehydes, ketones, alcohols, acids, furans and other compounds decreased with time, while the contents of phenols, aromatics and pyrazines increased in the middle stage of fermentation, but decreased slightly after fermentation. Esters and hydrocarbons decreased significantly in the middle stage of fermentation, and increased again in the late stage of fermentation. Heterocyclic compounds and aldehyde compounds in unfermented raw materials contribute greatly to the flavor of the compound liquid, while furan and pyrazine compounds are produced in the process of enzymatic hydrolysis and heating, which are flavor substances with baking aroma and are the products of Maillard reaction. Details are shown in table 2 below.

TABLE 2

| | | Absolute content (μg/100 mL) | | | |
|---|---|---|---|---|---|
| | | | Medium | After | Finished |
| | CAS | Unfermented | fermentation | fermentation | product |
| Aldehydes | | 1 | 9 | 9 | 9 |
| N-octanal | 124-13-0 | 1.847 | 1.532 | 0.485 | 0.707 |
| Nonanal | 124-19-6 | 8.475 | 8.992 | 1.946 | 2.293 |
| Decanal | 112-31-2 | 1.932 | 0.933 | 0.742 | 0.570 |
| Phenyl aldehyde | 100-52-7 | 0.248 | 0.970 | 1.805 | 3.468 |
| 2-butyl-2-octenal | 13019-16-4 | 0.979 | 0.612 | 0.279 | 0.181 |
| N-hexanal | 66-25-1 | 0.140 | 0.121 | — | 0.597 |
| Trans-2-nonanal | 18829-56-6 | 1.242 | 0.575 | — | — |
| Trans-2-decenal | 3913-81-3 | 0.561 | 0.524 | — | — |
| Trans-2-octenal | 2548-87-0 | 0.582 | 0.438 | — | — |
| 2,4-decadienal | 2363-88-4 | 0.741 | — | — | — |
| (E)-2-heptene | 18829-55-5 | 0.929 | — | — | — |
| Aldehyde | | | | | |
| Pentanal | 110-62-3 | — | — | — | 0.601 |
| 2,5- | 5779-94-2 | — | — | 0.868 | 1.533 |
| dimethylbenzaldehyde | | | | | |
| 4-N-propyl | 28785-06-0 | — | — | 0.845 | 0.336 |
| benzaldehyde | | | | | |
| 4-methoxy-2- | 52289-54-0 | — | — | 0.738 | — |
| methylbenzaldehyde | | | | | |
| Isovaleraldehyde | 590-86-3 | — | — | 0.637 | — |
| ketone | | 9 | 5 | 5 | 5 |
| Secondone | 111-13-7 | 6.086 | 4.877 | 2.410 | 1.587 |
| 2(5H)-furanone | 497-23-4 | 0.611 | 0.461 | 0.330 | 0.229 |
| 3-octanone | 106-68-3 | 0.485 | 0.606 | 0.336 | 0.149 |
| 2-nonanone | 821-55-6 | 0.487 | 0.512 | 0.364 | 0.221 |
| 2-undecanone | 112-12-9 | — | 0.646 | 0.444 | 0.631 |
| 2-heptanone | 110-43-0 | 2.945 | — | — | — |
| 2,3-octanedione | 585-25-1 | 0.929 | — | — | — |
| 6-methenyl spiro | | 0.720 | — | — | — |
| [4.4] nonane-1- | | | | | |
| one | | | | | |
| 4-ethyl-3-ethynyl- | 109273-68-9 | 0.673 | — | — | — |
| (9CI)2(5H)- | | | | | |
| furanone | | | | | |
| 4,4-dimethyl-1,5- | 33698-65-6 | 0.550 | — | — | — |
| hexadiene-3-one | | | | | |
| Esters | | 5 | 2 | 4 | 8 |
| Diisobutyl | 84-69-5 | 1.337 | — | 2.997 | 0.811 |
| Phthalate | | | | | |
| Dibutyl phthalate | 84-74-2 | 0.715 | — | 1.533 | 0.624 |
| 2,2,4-trimethyl- | 6846-50-0 | 0.429 | — | 0.738 | — |
| 1,3-pentanediol | | | | | |
| diisobutyrate | | | | | |
| 4-pentylbutane- | | 0.285 | 0.236 | — | 1.164 |
| 4-lactone | | | | | |
| N-hexyl formate | 629-33-4 | 4.076 | — | — | — |
| Amyl formate | 638-49-3 | — | 0.564 | — | — |
| Ethyl caproate | 123-66-0 | — | — | 0.130 | 0.758 |
| Ethyl decanoate | 110-38-3 | — | — | — | 1.791 |
| Ethyl butyrate | 105-54-4 | — | — | — | 3.285 |
| Linalyl formate | 115-99-1 | — | — | — | 0.599 |
| etheyl octanoat | 106-32-1 | — | — | — | 13.115 |
| Alcohol | | 3 | 6 | 4 | 2 |
| N-heptanol | 111-70-6 | 9.828 | 4.856 | 1.132 | 1.125 |
| N-octyl alcohol | 111-87-5 | 2.594 | 1.801 | 0.580 | — |
| Hexyl alcohol | 111-27-3 | — | 2.437 | 0.903 | — |
| Linalool | 78-70-6 | — | — | 0.591 | — |
| Furfuryl alcohol | 98-00-0 | 3.226 | 2.749 | — | — |
| (E)-3-(4- | | — | 0.549 | — | 0.206 |
| acetoxyphenyl) | | | | | |
| propen-1-ol | | | | | |
| 1-octene-3-ol | 3391-86-4 | — | 1.076 | — | — |
| acids | | 4 | 4 | 6 | 6 |
| N-hexanoic acid | 142-62-1 | 2.686 | 2.711 | 0.354 | 1.352 |
| caprylic acid | 124-07-2 | 0.651 | 0.463 | 0.139 | 0.123 |
| pelargonic acid | 112-05-0 | 1.283 | 1.045 | 0.340 | 0.378 |
| acetic acid | 64-19-7 | 1.116 | 0.732 | — | — |
| hydrocarbon | | 3 | 2 | 2 | 2 |
| hendecane | 1120-21-4 | 0.824 | 0.543 | 0.597 | 0.553 |
| hexadecane | 544-76-3 | 1.690 | 0.099 | — | 0.863 |
| Pentadecane | 629-62-9 | 0.243 | — | 0.598 | — |
| phenols | | 1 | 2 | 1 | 1 |
| 2,6-di-tert-butyl- | 128-37-0 | 0.163 | 0.150 | 0.216 | 3.949 |
| p-cresol | | | | | |
| 1,2,3,3A,4,5,6,8 | 89-88-3 | — | 0.521 | — | — |
| A-octahydro- | | | | | |
| 4.8 dimethyl-2- | | | | | |
| (1-methylethylene)- | | | | | |
| 6-chlorophenol | | | | | |
| Aromatic | | 1 | 2 | 1 | 2 |
| ortho-xylene | 95-47-6 | — | 0.667 | — | 2.041 |
| toluene | 108-88-3 | 0.440 | 1.687 | 1.478 | 3.285 |
| Furan compound | | 2 | 2 | 2 | 2 |
| 2-acetyl furan | 1192-62-7 | 1.109 | 0.994 | 1.239 | 0.804 |
| 2-pentylfuran | 3777-69-3 | 13.210 | 3.723 | 1.106 | 0.296 |
| Pyrazine | | 4 | 4 | 4 | 4 |
| compound | | | | | |
| ligustrazine | 1124-11-4 | 0.413 | 0.396 | 0.650 | 0.380 |
| 2- | 109-08-0 | 1.021 | 0.956 | 1.040 | 0.904 |
| methylpyrazine | | | | | |
| 2,6- | 108-50-9 | 0.659 | 0.907 | 0.732 | 0.749 |
| dimethylpyrazine | | | | | |
| 2,3,5- | 14667-55-1 | 0.967 | 0.924 | 0.685 | 0.641 |
| trimethylpyrazine | | | | | |
| other | | 2 | 2 | 2 | 2 |
| Phenylacetonitrile | 140-29-4 | 0.762 | 0.697 | 0.661 | 0.632 |
| 1,2,3,4,6,8α- | 16728-99-7 | — | 1.443 | 1.256 | 1.101 |
| hexahydro-1- | | | | | |
| isopropyl-4,7- | | | | | |
| dimethylnaphthalene | | | | | |
| 30.18 CADINA- | 38758-02-0 | 1.557 | — | — | — |
| 1,4- | | | | | |
| DIENE<TRANS−> | | | | | |

Note:
substance with absolute content below 0.5 μg/mL is not shown, and — means that the substance is not detected.

Many odorous substances (e.g. furanone, p-cresol, and a range of alkyl pyrazines) have become key players in fermentation, such as cocoa or milk chocolate; yet, furanone shows a downward trend during the fermentation process. Maillard reaction occurs during the production of this ready-to-combine liquid to transform the flavor of the raw materials, with products including pyrazine and furan compounds; pyrazine compounds are cyclic compounds containing nitrogen, which have important flavor characteristics; four pyrazine compounds and two furan compounds are generated during the fermentation process of beverage, where pyrazine compounds undergo fermentation without change in species, but their relative content increases and their threshold is low, usually with pleasant nutty, barbecue and other odors; one of the more increased ligustrazine is an active alkaloid that can be produced by the Maillard reaction of 3-hydroxy-2-butanone (ethyl coupling) in a fermentation system, mainly from the conversion of amino acids to ammonia, and modern medical research has proven that ligustrazine has the ability to improve cerebral ischemia, improve microcirculation, anti-platelet aggregation and prevent thrombosis; the followed increase is 2,6-dimethylpyrazine which also proved to be a key aroma compound in Boletus edulis. As such, pyrazine compounds also contribute significantly to the flavor of beverages. However, little has been found about the fermentation of edible fungi to increase alkylpyrazines, and their exact biosynthetic pathways and key enzymes remain unclear.

One substance decreased and one substance increased in furans. 2-pentylfuran, a typical off-flavor substance, is reduced from 13.210 μg/mL to 1.106 μg/mL, and its reduction by fermentation facilitates the beverage to be accepted by consumers. 2-acetylfuran, which has sweet almond and creamy taste, is increased by fermentation to improve the flavor of the beverage.

Aldehydes are the most detected compounds with low odor thresholds and strong odor properties; most aldehydes have fruity, fatty and nutty flavors; pre-fermentation aldehydes are mainly generated by fat oxidation and generally occur during the preparation of raw materials or during enzymatic digestion; a total of 17 aldehydes are detected during the fermentation process, accounting for a relatively large proportion of the fermented beverage; as fermentation proceeds, the content of most aldehydes decreases, and the concentration of many typical flavor substances is reduced, including n-octanal, n-hexanal, nonanal, decanal, 2,4-decadienal, trans-2-nonanal, etc.; these substances with green odor are reduced to make the beverage more acceptable to consumers; however, the mechanism of degradation by edible bacteria is not fully understood.

A few aldehydes are first increasing in content and then gradually decreasing, or keep increasing, or even newly formed, such as benzaldehyde and isovaleraldehyde; among them, benzaldehyde is an aromatic aldehyde produced by the metabolism of aromatic amino acids and benzoic acid during the fermentation process, which has a sweet and fruity flavor and is considered a safe food additive and flavoring substance in the US and EU respectively; and isovaleraldehyde has a pleasant fruit aroma at low concentrations and significantly contributes to the flavor of beverages.

Alcohols have a high threshold, so if they are not present in very high concentrations or an unsaturated form, their contribution to food flavor is insignificant; however, despite their high threshold, alcohols can react with acids to form esters that promote the formation of flavor after fermentation. The esters are important volatile flavor substances in the fermentation process with lower threshold values than alcohols, mainly from condensation reactions generated by alcohols and acids, enzymatic reactions, or hydrolysis of fatty acids in the product and metabolism during the fermentation of strains. In the four stages of beverage fermentation (unfermented, during fermentation, after fermentation, and finished product), the relative content of alcohols and acids decreases after fermentation, while the relative content of esters, which endow the beverage with a sweet and fruity aroma, increases after fermentation. Only three acids are detected after fermentation, and their contents are low and the threshold value of acids produced during fermentation is relatively higher, which therefore provide a little overall contribution to the flavor of fermented beverages. Some unsaturated alcohols produced during fermentation impact flavor, such as linalool, which has a floral odor and is a known component of the aroma of many other fungi (e.g., lentinus edodes, Trametes versicolor, etc.).

During fermentation, microorganisms reduce certain types of aldehydes to alcohols or oxidize them to organic acids, releasing bound phenolic compounds. 2,6-di-tert-butyl-p-cresol as well as toluene in aromatic compounds increase in content as fermentation proceeds during the fermentation of Agrocybe cylindracea. 2,6-di-tert-butyl-p-cresol and toluene have a certain contribution to the flavor of the beverage, where 2,6-di-tert-butyl-p-cresol is a frequently used phenolic antioxidant with a low threshold value, and toluene has a floral aroma.

2-undecanone, which has a milky fruit flavor and has been shown to significantly reduce DNA damage and inflammation and thereby prevent tumorigenesis, is detected only in the middle and late fermentation stages and in the finished beverage. Other ketones and hydrocarbons are found in the unfermented compound liquid, and most of them have a decreasing trend after fermentation. It is possible that they are produced from the raw materials through the thermal degradation process, and the odor threshold concentration of hydrocarbons and ketones is high, whose overall contribution to the flavor of fermented beverages is not significant.

Then the free amino acids in the beverage are analyzed by an automatic amino acid analyzer, with specific methods as follows:

1 mL of supernatant is placed in a centrifuge tube, and added with 9 mL of 2% sulfosalicylic acid, followed by mixing well standing for 15 min, and centrifuging at 6,000 r/min for 10 min; then the supernatant after centrifuging is taken to pass through a 0.45 micrometer (μm) membrane and tested, where the amino acid content is quantified by standard external method; Results are shown in Table 3 below.

TABLE 3

| | | During | After | Finished |
|---|---|---|---|---|
| | Unfermented | fermentation | fermentation | product |
| Amino acid types | mg/100 g | mg/100 g | mg/100 g | mg/100 g |
| Aspartic acid | 25.5 | 23.46 | 15.3 | 16.32 |
| Threonine* | 6.12 | 8.16 | 10.2 | 9.18 |
| Serine | 7.14 | 9.18 | 11.22 | 11.22 |
| Glutamic acid | 275.4 | 262.14 | 247.86 | 249.9 |
| Glycine | 13.26 | 13.26 | 17.34 | 16.32 |
| Alanine | 34.68 | 38.76 | 43.86 | 41.82 |
| Valine* | 13.26 | 12.24 | 15.3 | 14.28 |
| Methionine* | 12.24 | 3.06 | 4.08 | 4.08 |
| Isoleucine* | 18.36 | 12.24 | 10.2 | 14.28 |
| Leucine* | 14.28 | 18.36 | 18.36 | 22.44 |
| Tyrosine | 13.26 | 0 | 11.22 | 11.22 |
| Phenylalanine* | 15.3 | 24.48 | 16.32 | 20.4 |
| Histidine | 11.22 | 9.18 | 9.18 | 10.2 |
| Lysine* | 6.12 | 10.2 | 11.22 | 11.22 |
| Arginine* | 12.24 | 22.44 | 27.54 | 25.5 |
| Proline | 104.04 | 95.88 | 113.22 | 97.92 |
| Total | 582.42 | 563.04 | 582.42 | 576.3 |
| Essential amino | 97.92 | 111.18 | 113.22 | 121.38 |
| acid content | | | | |
| Essential amino | 16.81% | 19.75% | 19.44% | 21.06% |
| acid % | | | | |

As the main precursors of volatile flavors, free amino acids are substances that have a large impact on the taste of foods and directly or indirectly affect the flavor of foods. Based on their taste characteristics, amino acids are classified to be fresh (e.g., glutamic acid, aspartic acid), bitter (e.g., valine, arginine, leucine, histidine, methionine, phenylalanine and isoleucine) and sweet (e.g., threonine, glycine, serine and alanine).

After fermentation, the amino acids of fresh taste and some of bitter taste have decreased, but the amino acids of sweet taste have all increased, and the content of fresh taste amino acids is still high despite the decrease; among them, amino acids of sweetness, bitterness and freshness are the most important components of the taste of fermented beverage, although the content of some bitter amino acids has increased, most of the amino acids presenting bitterness do not have taste activity and the bitterness is easily covered by freshness and sweetness, which enables the beverage to present a strong taste of freshness and sweetness. Additionally, these amino acids interact with other taste-presenting substances to have a refreshing effect, thus making the beverage complex in flavor. Therefore, the fermentation treatment changes the ratio of amino acids in the beverage and contributes to the improvement of flavor.

The total amount of amino acids in the sample after fermentation is 582.42 mg/100 g, and the total amount of essential amino acids is 113.22 mg/100 g, accounting for 19.44% of the total free amino acids. As the fermentation process of the beverage proceeds, the total amount of free amino acids in the beverage shows a trend of first decreasing and then increasing, and the content of all the essential amino acids rises, except for the content of methionine and isoleucine, which are decreased. The overall content firstly increased and then remained stable, and the ratio of essential amino acids to total free amino acids showed an upward trend and then a downward trend.

The reason why the total amount of free amino acids decreases first during the fermentation process could be due to the imperfect metabolism of the proteins of agrocybe cylindracea, where some of the free amino acids in the beverage are utilized; differences in the content of different amino acids are caused by their transformation by the strains during the fermentation process. It could be seen from the tables that fermented compound liquid has the highest content of glutamic acids, whereas fermented compound liquid with high content of glutamic acid is the best substrate for the production of fermented beverage with high γ-aminobutyric acid (GABA) content; the decrease in glutamic acid during fermentation is probably caused by the Glutamic Acid Decarboxylase (GAD) activity of the Agrocybe cylindracea during fermentation, which catalyzes the decarboxylation of glutamic acid to synthesize GABA. The fermented sample contains an increased amount of lysine and arginine, where lysine promotes growth and development of human body, and arginine plays an important role as a component of the nitric oxide (NO) production pathway, which contributes to vasodilation of arterial and venous vessels and may have a therapeutic effect on patients with hypertension.

In conclusion, although the total amount of amino acids after fermentation is the same as that before fermentation, the amount of each type of amino acid varies during fermentation. Considering that a balanced and sufficient intake of amino acids is the basic premise of human health, the beverage provided by the present embodiment meets people's demand for protein food with such a high content of amino acids. Any increase or decrease in amino acid content in the finished product is supposed to be the result of added excipients or sterilization.

Embodiment 2 Different edible fungi fermented kudzuvine root and coix seeds compound liquid
(1) Same as step (1) of Embodiment 1;
(2) same as step (2) of Embodiment 1;
(3) same as step (3) of Embodiment 1;
(4) same as step (4) of Embodiment 1;
(5) inoculating edible fungus seed solution into the sterilized compound liquid with an inoculation amount of 4%, and carrying out constant temperature fermentation in a shaking table with a rotation speed of 170 r/min, fermentation temperature of 27° C. and fermentation duration of 3 days, obtaining a fermentation broth; and
(6) following the fermentation, obtaining the edible fungi fermented kudzuvine root and coix seeds compound liquid.

The kudzuvine root and coix seeds compound liquids fermented by different edible fungi of the present embodiment are evaluated in terms of GABA content and sensory score with results as shown in Table 4.

TABLE 4

| | GABA Content | Sensory |
|---|---|---|
| Strains | mg/100 ml | score |
| Poria cocos | 1.90 ± 0.17^{a} | 60.48 ± 1.56^{c} |
| agrocybe cylindracea | 2.07 ± 0.06^{a} | 74.32 ± 2.97^{a} |
| Grifola frondosa | 1.14 ± 0.00^{b} | 64.56 ± 1.67^{b} |
| Lentinus edodes | 2.03 ± 0.03^{a} | 64.68 ± 3.07^{b} |
| Pleurotus ostreatus | 0.88 ± 0.09^{c} | 71.72 ± 3.12^{a} |
| Saccharified liquid of kudzuvine | 1.91 ± 0.15^{a} | 60.28 ± 4.62^{c} |
| root and coix seeds | | |

Note:
different letters in the same column indicate significant differences (P < 0.05), while the same letters indicate no significant differences (P > 0.05).

It can be seen from Table 4 that the GABA contents of Pleurotus ostreatus and Grifola frondosa fermented compound liquid are significantly decreased after fermentation. Therefore Agrocybe cylindracea is finally selected as the subsequent fermentation strain by comparing the GABA content and sensory score after fermentation.

Embodiment 3 Agrocybe cylindracea fermented kudzuvine root and coix seeds compound liquid with different proportions
(1) Same as step (1) of Embodiment 1;
(2) same as step (2) of Embodiment 1;
(3) mixing the above-mentioned saccharified liquids of kudzuvine root and germinated coix seeds according to different volume ratios to obtain compound liquids;
(4) same as step (3) of Embodiment 1.
(5) inoculating edible fungus seed solution into the sterilized compound liquids with an inoculation amount of 4%, and carrying out constant temperature fermentation in a shaking table with a rotation speed of 170 r/min, fermentation temperature of 27° C. and fermentation duration of 3 days, obtaining a fermentation broth; and
(6) following the fermentation, obtaining the Agrocybe cylindracea fermented kudzuvine root and coix seeds compound liquids.
the kudzuvine root and coix seeds compound liquids fermented by agrocybe cylindracea of different volume ratios of the present embodiment are determined in terms of proportions with results as shown in Table 5.

TABLE 5

| Saccharified liquid of | | Protein | | |
|---|---|---|---|---|
| kudzuvine root:saccharified | Sensory | content | Coixol | Puerarin |
| liquid of germinated coix seeds | score | mg/100 mL | ug/100 mL | ug/mL |
| 1:9 | 69.00 ± 1.84^{c} | 35.43 ± 0.97^{a} | 67.15 ± 0.41^{a} | 14.77 ± 0.09^{d} |
| 3:7 | 69.24 ± 3.1^{c} | 35.06 ± 0.08^{a} | 48.9 ± 1.12^{b} | 45.06 ± 1.23^{c} |
| 1:1 | 74.32 ± 2.97^{a} | 32.82 ± 0.21^{b} | 45.48 ± 0.06^{c} | 76.53 ± 0.71^{e} |
| 7:3 | 72.15 ± 2.7^{ab} | 29.79 ± 0.08^{c} | — | 111.15 ± 2.14^{b} |
| 9:1 | 70.98 ± 3.47^{bc} | 28.91 ± 0.16^{d} | — | 146.68 ± 3.24^{a} |

As can be seen from Table 5, after the saccharified liquids of kudzuvine root and germinated coix seeds are mixed in different proportions for fermentation, it is found by sensory observation that the fermentation broths in the ratio of 1:9 and 3:7 have obvious stratification and the aroma of kudzu is weak, where the fermentation broth with ratio of 9:1 has a heavy smell of herbal medicine and a bitter taste, while the fermentation broth with ratio of 7:3 has weak aroma of coix seeds and strong aroma of kudzuvine root, which covers up the flavor of coix seeds, making the fermented beverage taste incongruous and unpleasant; also, the content of protein is gradually reduced as the saccharified liquid of coix seeds is reduced, and the content of coixol is diluted and too low to be detected. The fermentation broth mixed in the ratio of 1:1 has a delicate taste, with both the unique aroma of kudzuvine root and the sweetness of coix seeds, and moderate taste and uniform tissue state. Therefore, the optimal ratio of saccharified liquids of coix seeds and kudzuvine root for preparing the fermented beverage is determined as 1:1 based on the sensory score, soluble protein content, coixol content and puerarin content.

Embodiment 4 Single-factor experiment on fermentation technology of kudzuvine root and coix seeds compound liquid fermented by Agrocybe cylindracea
(1) Same as step (1) of Embodiment 1;
(2) same as step (2) of Embodiment 1;
(3) same as step (3) of Embodiment 1;
(4) same as step (4) of Embodiment 1;
(5) inoculating Agrocybe cylindracea seed solution into the sterilized compound liquid by taking inoculation amount, rotating speed of shaking table, fermentation temperature and fermentation duration as single factor variables, respectively, to obtain Agrocybe cylindracea fermented kudzuvine root and coix seeds compound liquids.

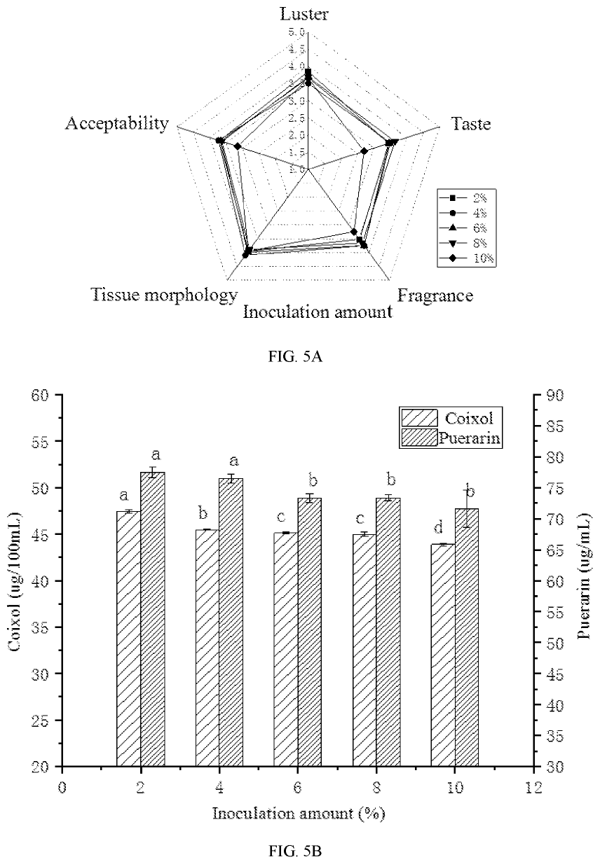

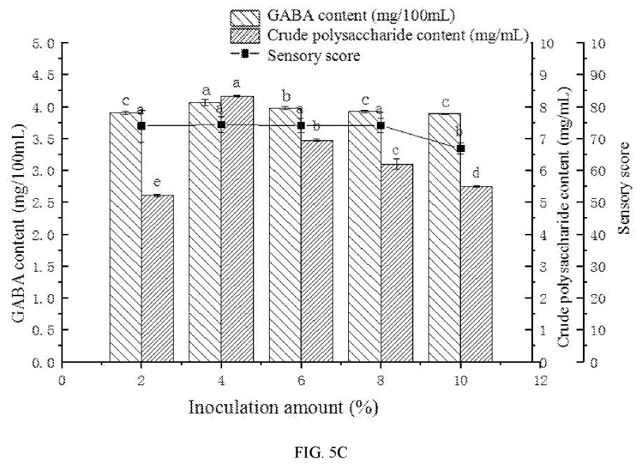

The influence of different inoculation amounts on sensory score, puerarin, coixol, GABA and polysaccharide contents of Agrocybe cylindracea fermented kudzuvine root and coix seeds compound liquid are shown in FIG. 5A-FIG. 5C. The GABA content and crude polysaccharide content first increase and then decrease when the inoculation amount gradually increases, and the highest GABA content and crude polysaccharide content are found when the inoculation amount is 4%. GABA production by fermentation is based on the action of decarboxylase in fermenting microorganisms on glutamic acid in the medium, and germinated coix seeds contain more free amino acids, which provide the basis for GABA production by fermentation of Agrocybe cylindracea. In addition, Agrocybe cylindracea needs to be supplied with sufficient oxygen for both growth and accumulation of bioactive substances such as polysaccharides. During the fermentation process with a fixed inoculation amount, a smaller inoculation amount ensures the aeration required for the growth of Agrocybe cylindracea, which results in higher polysaccharide and GABA contents. However, an excessive inoculation amount in a medium with the same nutrient composition and content may result in an insufficient supply of nutrients required by the mycelium for better polysaccharide accumulation, leading to a decrease in polysaccharide content. The contents of coixol and puerarin are decreased as the amount of inoculation amount increases, which is probably caused by the consumption during the growth of Agrocybe cylindracea. Sensory evaluation declines severely when the inoculation amount is relatively large, probably due to the shortened fermentation cycle, excessive fermentation, excessive bacteriophages, therefore the acceptability decreases and the sensory quality declines; therefore, the inoculation amount of 4%-6% is selected based on a comprehensive consideration.

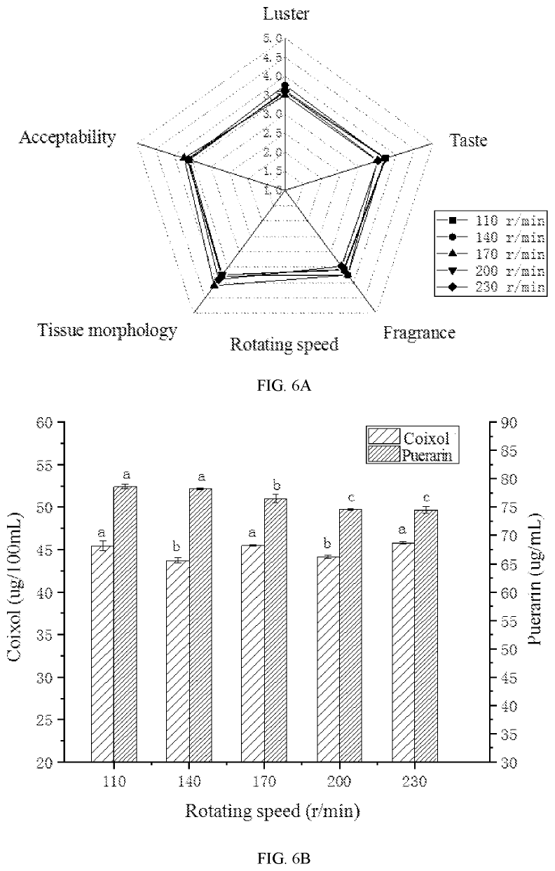

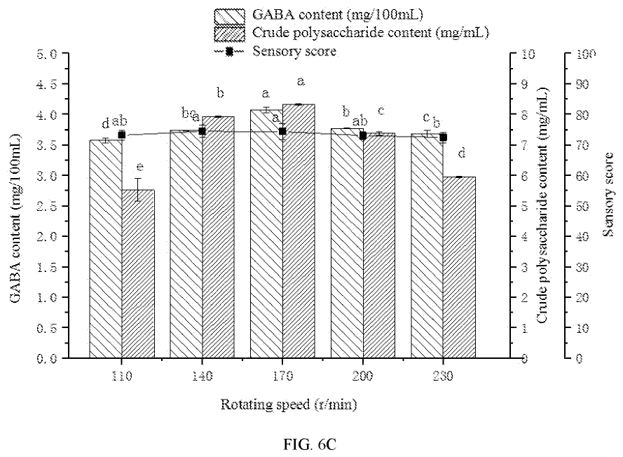

See FIG. 6A-FIG. 6C for the influence of different shaking speed on sensory score, puerarin, coixol, GABA and polysaccharide content of Agrocybe cylindracea fermented kudzuvine root and coix seeds compound liquid. It can be seen from the drawings that the contents of GABA and crude polysaccharides are increased along with the increase of rotating speed, since increased rotating speed leads to an increase in aeration and dissolved oxygen. The highest crude polysaccharide content and GABA content are measured at a rotating speed of 170 r/min, and the contents of crude polysaccharide and GABA at a rotating speed of 140 and 200 r/min are both decreased. In the fermentation process, the rotational speed directly affects the oxygen transfer and dissolution, and excessive high and low dissolved oxygen content in the fermentation process will affect the growth of mycelium and the accumulation of polysaccharides. Under the condition of same amount of liquid loading, the rotating speed either too high or too low is not conducive to the growth of the bacterium, for too low a rotating speed leads to low amount of dissolved oxygen in the fermentation flask, and the mycelium fails to be fully exposed to oxygen, while too high a rotating speed will produce additional metabolites and foam, which will inhibit the contact between oxygen and liquid and also damage the mycelium; in this experiment, the dissolved oxygen of the fermentation broth is most favorable to the accumulation of polysaccharides in the mycelium of Agrocybe cylindracea under the condition that the rotating speed of shaking bed is 170 r/min, with the content of polysaccharides being the highest; the content of puerarin decreases along with the increase of rotating speed, and the content of coixin shows fluctuating changes with no significant changes in sensory; based on comprehensive consideration, the rotating speed of 140-200 r/min is selected.

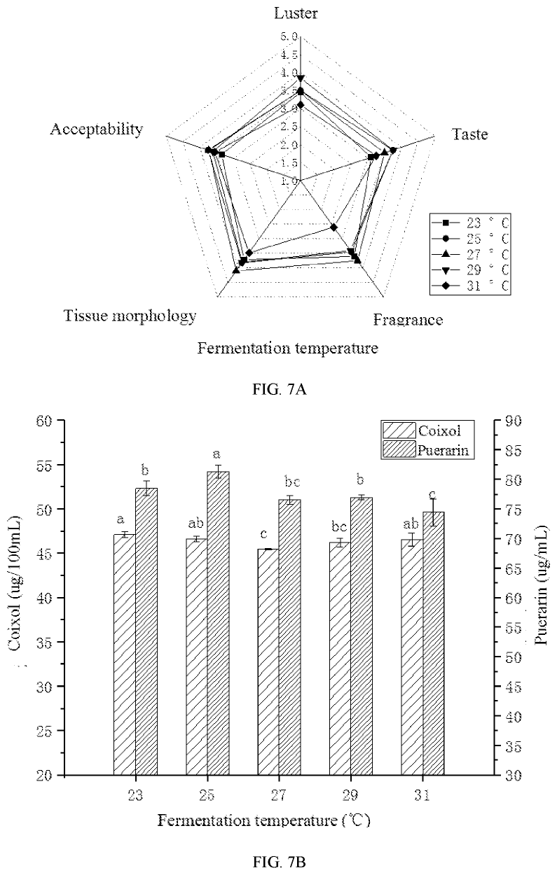

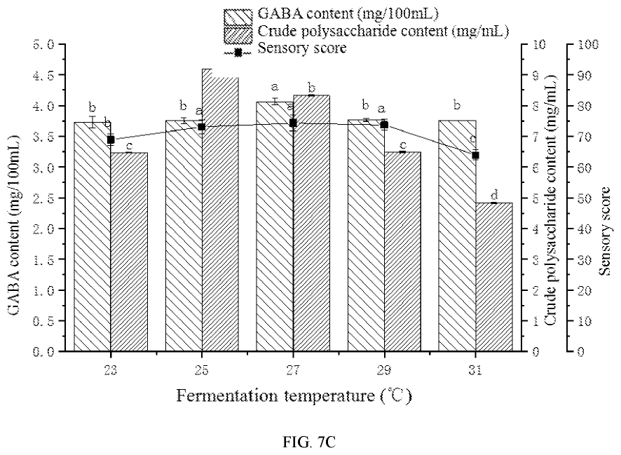

See FIG. 7A-FIG. 7C for the influence of different fermentation temperatures on sensory score, puerarin, coixol, GABA and polysaccharide contents of Agrocybe cylindracea fermented kudzuvine root and coix seeds compound liquid. It is shown that the mycelium of Agrocybe cylindracea can grow in a wide temperature range of 23° C.-31° C.; as the temperature of fermentation increases, the GABA content and polysaccharide production in the fermentation broth are increased continuously, with the highest polysaccharide content measured at 25° C. and the highest GABA content at 27° C., indicating that the Agrocybe cylindracea is better to be fermented at temperature above 25° C.; as the temperature of fermentation increases, there is a trend of increasing and then decreasing of both polysaccharide and GABA contents. No significant difference in sensory evaluation is observed in the temperature range of 25-29° C. The content of coixol firstly decreases and then increases as the temperature increases, but the change is not significant, probably due to the increase of temperature during the fermentation process, which makes coixol leach out; the content of puerarin gradually decreases as the temperature increases. Considered comprehensively, the temperature range of 25-29° C. is determined.

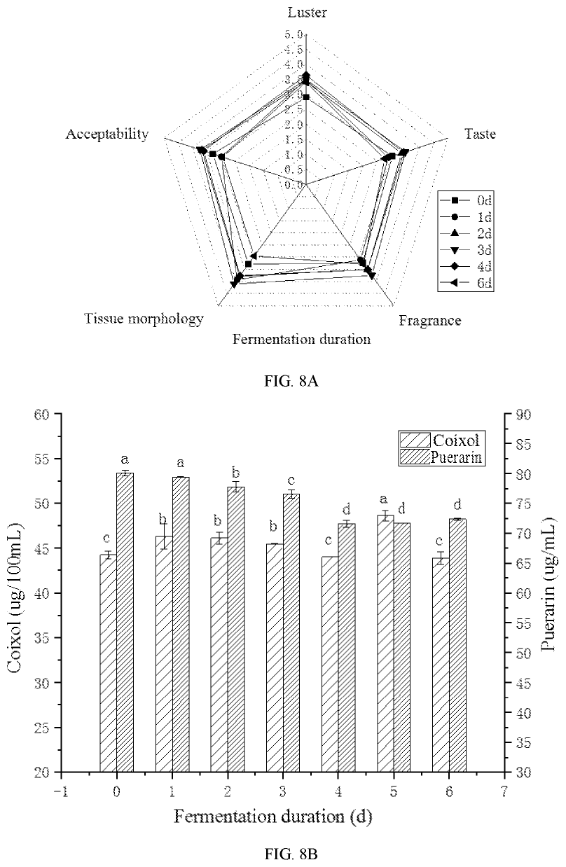

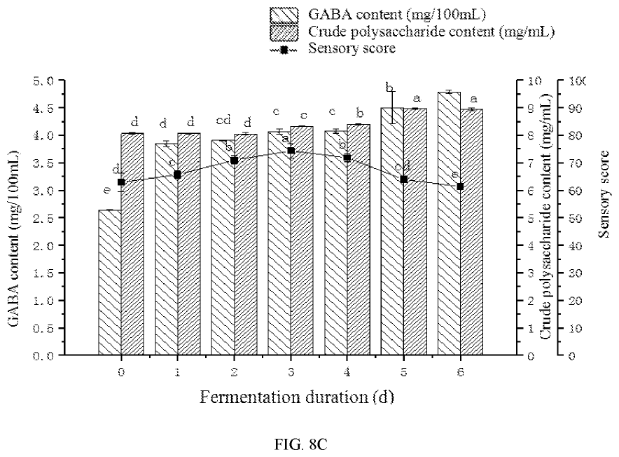

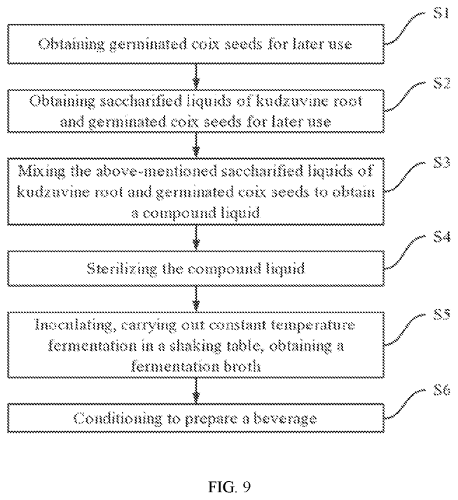

FIG. 8A-FIG. 8C show the influence of different fermentation duration (0-6 days) on the sensory score, puerarin, coixol, GABA and polysaccharide content of Agrocybe cylindracea fermented kudzuvine root and coix seeds compound liquid; it can be seen from the drawings that with the increase of fermentation duration, the polysaccharide and GABA contents in the broth gradually increase and reach the maximum at the 6th days; polysaccharides provide health care value to the beverage, including protecting the organism from oxidative stress and reducing fat deposition, a variety of physiological functions are also associated with GABA. As a result, it is suggested that the bioactivity of kudzuvine root coix seeds compound liquid is improved after being fermented by Agrocybe cylindracea.

Sensory scores after fermentation show a trend of increasing and then decreasing, and optimum sensory scores are obtained on the third day; the sensory scores gradually decrease with the extension of fermentation duration, where the luster and aroma of Agrocybe cylindracea fermented kudzuvine root and coix seeds compound liquid is excellent in the early stage of fermentation; yet, as the fermentation proceeds, the mycelium gradually increases in volume and size, resulting in poor fluidity and reduced acceptability; also, the content of puerarin decreases as the fermentation duration increases, which, remains to be further studied, may be the result of its conversion into other bioactive substances during the fermentation of edible fungi; the content of coixol fluctuates as the fermentation duration increases; therefore, the fermentation duration is determined to be of 2-4 days on the basis of comprehensive consideration.

The fermentation process conditions such as inoculation amount, fermentation temperature, fermentation duration and rotating speed are optimized using response surface based on the results of single-factor test, and the sensory score, GABA content and crude polysaccharide content are used as response values to optimize the parameters of each factor for the agrocybe cylindracea fermented kudzuvine root and coix seeds beverage and determine the best conditions of the fermentation process, and the conditions include: inoculation amount of 5.02%, rotating speed of 164.8 r/min, fermentation temperature of 26.41° C., and fermentation duration of 3.42 days; under such optimized conditions, the predicted value of crude polysaccharide, GABA and sensory score is 8.84 mg/mL, 4.27 mg/100 mL and 81.95 respectively. A specific revision of the optimized conditions for the fermentation process of the beverages is carried out to examine the accuracy of the results of the response surface optimization test and for the convenience of the operation at a later stage, including: an inoculation amount of 5%, rotating speed of 165 r/min, fermentation temperature of 26.5° C. and fermentation duration of 3.5 days. Three independent tests are conducted under these fermentation conditions, and the crude polysaccharide content of the beverage at the best quality is 8.41 mg/mL, the content of GABA is 4.2 mg/100 mL, the sensory score is 81.36, which are close to the predicted results of the response surface, indicating that the beverage fermentation process obtained in this experiment is reliable.

Embodiment 5 Taste conditioning and stabilizer screening of kudzuvine root and coix seeds beverage fermented by Agrocybe cylindracea
(1) Same as step (1) of Embodiment 1;
(2) same as step (2) of Embodiment 1;
(3) same as step (3) of Embodiment 1;
(4) same as step (3) of Embodiment 1;
(5) same as step (3) of Embodiment 1; and
(6) conditioning the taste of the beverage, screening the stabilizer and obtaining the beverage after the fermentation is finished.

See Table 6 for the sensory scores of various excipients at different additions.

TABLE 6

| Addition of | Sensory | Added amount of | Sensory | Added amount of non- | Sensory |
|---|---|---|---|---|---|
| citric acid (%) | score | white sugar (%) | score | dairy creamer (%) | score |
| 0.1 | 73.36 ± 3.88 | 0.5 | 79.92 ± 3.71 | 1 | 81.60 ± 1.57 |
| 0.2 | 72.88 ± 2.37 | 1 | 81.04 ± 2.65 | 2 | 80.72 ± 2.12 |
| 0.3 | 71.44 ± 2.54 | 1.5 | 81.84 ± 3.05 | 3 | 80.48 ± 2.63 |
| 0.4 | 71.36 ± 2.59 | 2 | 81.20 ± 2.47 | 4 | 80.32 ± 3.71 |
| 0.5 | 68.72 ± 3.22 | 2.5 | 80.24 ± 3.19 | 5 | 80.32 ± 2.47 |

Taking sensory score as an index, the taste of fermented beverage of kudzuvine root and coix seeds conditioned with citric acid, white sugar and non-dairy creamer being added, respectively.

Citric acid is an edible acid additive with good solubility and is wholly dissolved in beverages, making the sensory of beverages better and enabling an appetizing effect. However, the sensory score gradually decreases when citric acid is added to the product in this experiment, with the sensory score of a minimum content of citric acid lower than the initial score, indicating that the citric acid is not applicable in this experiment, and therefore citric acid is not added to the subsequent product.

White sugar is a relatively common excipient in beverages, providing multiple effects such as improving the taste and texture of the beverage, serving as a source of energy, and improving the rheological properties of the beverage. However, as grain starch already contains sugar after liquefaction and saccharification, adding too much sugar to this beverage makes the taste cloying and may even cause thirst and gaining weight. It can be seen from Table 6 that the sensory score increases and then decreases as the sugar content increases, and the highest sensory score of 81.84 is obtained at 1.5% addition.

Non-dairy creamer is a milky white powdered solid with good solubility and a soft, creamy taste. It is often used to whiten beverages such as coffee, cocoa and tea to soften the acidic taste and provide a desired flavor and texture. The fermented beverage is improved by adding non-dairy creamer to make the brown color of the beverage more acceptable to the public, and also to make the taste of the beverage more refreshing, smooth and pleasantly creamy. It is shown in Table 6 that the sensory score decreases with the increase of non-dairy creamer, so the amount of non-dairy creamer is decided to be 1%.

The centrifugal sedimentation rates of various stabilizers at different additions are shown in Table 7.

TABLE 7

| Added | | | | |
|---|---|---|---|---|
| amount | Centrifugal sedimentation rate (%) | | | |
| (%) | xanthan gum | pectin | sodium alginate | CMC-Na |
| 0 | 5.34 ± 0.05^{c} | 5.34 ± 0.05^{b} | 5.34 ± 0.05^{b} | 5.34 ± 0.05^{ab} |
| 0.05 | 4.05 ± 0.11^{d} | 4.41 ± 0.21^{c} | 4.45 ± 0.23^{c} | 4.96 ± 0.15^{d} |
| 0.1 | 5.58 ± 0.44^{c} | 5.32 ± 0.19^{b} | 5.61 ± 0.01^{b} | 5.12 ± 0.15^{c} |
| 0.15 | 6.20 ± 0.33^{b} | 5.34 ± 0.03^{b} | 5.68 ± 0.23^{b} | 5.26 ± 0.13^{bc} |
| 0.2 | 6.43 ± 0.07^{b} | 5.86 ± 0.08^{a} | 6.44 ± 0.35^{a} | 5.39 ± 0.08^{ab} |
| 0.25 | 7.26 ± 0.25^{a} | 6.23 ± 0.50^{a} | 6.45 ± 0.08^{a} | 5.48 ± 0.05^{a} |

The centrifugal sedimentation rate is used as an indicator to screen the suitable stabilizer for the fermented beverage. As can be seen from Table 7, the centrifugal sedimentation rates of four stabilizers gradually increase with an increasing amount of stabilizer, which may be due to the fact that stabilizers at low concentrations can increase the viscosity of the product, improve the stability of the beverage system and reduce the centrifugal sedimentation rate. However, if the content of stabilizers continues to increase, the viscosity of the system increases significantly, which makes some of the liquid stick in the centrifuge tube after centrifugation, resulting in inaccurate measurement; the centrifugal sedimentation rates of all four stabilizers were lower than those of no addition when the adding amount is 0.05%; the stabilizers at this concentration have better stability, with xanthan gum and pectin having lower sedimentation rates among the four stabilizers, so xanthan gum and pectin are considered to be compounded for subsequent experiments.

Orthogonal tests on white sugar, phytolipid powder, xanthan gum and pectin are conducted according to the principle of orthogonal experimental design with sensory score and centrifugal sedimentation rate as the indicators, and the best formulation of the process for the fermented beverage is 2% of white sugar, 1.5% of phytolipid powder, 0.05% of xanthan gum and 0.04% of pectin. The present application optimizes the fermentation process and conditioning process of Agrocybe cylindracea fermented kudzuvine root and coix seeds compound liquid with two indicators of sensory evaluation and functional composition, where the sensory evaluation includes luster, taste, fragrance, tissue morphology and acceptability, and the functional indicators include polysaccharide content and GABA content.

After factor exploration and optimization, the content of crude polysaccharide in the fermented beverage of kudzuvine root and coix seeds is increased from the original 8.067 mg/mL to 8.41 mg/mL, and that of GABA is increased from the original 2.64 mg/100 mL to 4.2 mg/100 mL. Polysaccharides are often used to enhance the health value of beverages due to their unique health care properties, and GABA also has a variety of physiological functions. The results show that after the fermentation of Agrocybe cylindracea, the biological activity of kudzuvine root and coix seeds fermented beverage is improved with a uniform coffee color and a strong fermentation flavor of tea tree mushroom, as well as the fragrance of coix seeds and the freshness of kudzuvine root, with no off-flavor; besides, the sensory quality of the fermented beverage is effectively improved.

The above-mentioned embodiments only describe the preferred mode of the application, but do not limit the scope of the application. On the premise of not departing from the design spirit of the application, all kinds of modifications and improvements made by ordinary technicians in the field to the technical scheme of the application shall fall within the scope of protection defined by the claims of the application.

1. A method for preparing Agrocybe cylindracea fermented kudzuvine root and coix seeds beverage, comprising:
step 1, germinating coix seeds to obtain germinated coix seeds;
step 2, pulping kudzuvine root with water into a paste of kudzuvine root, gelatinizing the paste of kudzuvine root, followed by adding high-temperature amylase for primary enzymolysis and inactivating enzyme, and adding glucoamylase for secondary enzymolysis and inactivating enzyme to obtain saccharified liquid of kudzuvine root; pulping germinated coix seeds with water to obtain a paste of germinated coix seeds, gelatinizing the paste of germinated coix seeds, followed by adding with high-temperature amylase for primary enzymolysis and inactivating enzyme, and adding with glucoamylase for secondary enzymolysis and inactivating enzyme to obtain saccharified liquid of germinated coix seeds; and
step 3, mixing the saccharified liquids of kudzuvine root and germinated coix seeds, followed by sterilizing and inoculating with Agrocybe cylindracea seed solution for fermentation, then obtaining a fermented beverage prepared using kudzuvine root, coix seeds and agrocybe cylindracea.

2. The method for preparing Agrocybe cylindracea fermented kudzuvine root and coix seeds beverage according to claim 1, wherein the germinated coix seeds are obtained by: subjecting coix seeds to germinating treatment in an environment with a temperature of 27-31 degree Celsius (° C.) and a humidity of 88-93 percent (%) for 22-28 hours (h).

3. The method for preparing Agrocybe cylindracea fermented kudzuvine root and coix seeds beverage according to claim 2, wherein the coix seeds are disinfected before subjecting to germinating treatment.

4. The method for preparing Agrocybe cylindracea fermented kudzuvine root and coix seeds beverage according to claim 1, wherein as preparing the saccharified liquid of kudzuvine root in step 2, the kudzuvine root is in a mass-volume ratio of 1 gram (g): 8-10 milliliters (mL) with water during a process of pulping into paste; the gelatinizing refers to gelatinizing at 85-95° C. for 25-35 min; the high-temperature amylase added is in a concentration of 180-220 micrograms (u/g); and the primary enzymolysis specifically comprises heating at 85-95° C. for 40-50 min; and
as preparing the saccharified liquid of germinated coix seeds in step 2, the saccharified liquid of germinated coix seeds is in a mass-volume ratio of 1 g: 8-10 mL with water during a process of pulping into paste; the gelatinizing refers to gelatinizing at 85-95° C. for 25-35 min; the high-temperature amylase added is in a concentration of 180-220 micrograms (u/g); and the primary enzymolysis specifically comprises heating at 85-95° C. for 40-50 min.

5. The method for preparing Agrocybe cylindracea fermented kudzuvine root and coix seeds beverage according to claim 1, wherein as preparing the saccharified liquids of kudzuvine root and germinated coix seeds in step 2, the glucoamylase is added in a concentration of 280-320 u/g; and the secondary enzymolysis includes heating at 60-70° C. for 75-85 min.

6. The method for preparing Agrocybe cylindracea fermented kudzuvine root and coix seeds beverage according to claim 1, wherein the saccharified liquids of kudzuvine root and germinated coix seeds are in a volume ratio of 3: 7-7:3.

7. The method for preparing Agrocybe cylindracea fermented kudzuvine root and coix seeds beverage according to claim 1, wherein the sterilizing in step 3 is carried out at 118-125° C. for 18-25 min.

8. The method for preparing Agrocybe cylindracea fermented kudzuvine root and coix seeds beverage according to claim 1, wherein the Agrocybe cylindracea seed solution is inoculated with an amount of 4-6 weight percentage (wt %); the fermentation is carried out under rotating speed of 150-190 rotations per minute (r/min) at fermentation temperature of 25-29° C. for a fermentation duration of 2-4 days; and the fermentation also comprises a step of adding auxiliary materials for conditioning.

9. The method for preparing Agrocybe cylindracea fermented kudzuvine root and coix seeds beverage according to claim 1, wherein the Agrocybe cylindracea seed solution is prepared as follows:
(1) slant culture of original seed: selecting mycelium block from master test tube, inoculating the mycelium block to slant culture medium, and culturing the medium under constant temperature until thallus grows all over the slant; and
(2) liquid seed culture: selecting mycelium block from the cultivated slant, inoculating the mycelium block into liquid seed culture medium, and homogenizing after culturing at a shaking table under constant temperature; inoculating the homogenized seed solution into the liquid seed culture medium in an inoculating amount of 3.5-4.5 wt %; and culturing at a shaking table under constant temperature to obtain the Agrocybe cylindracea seed solution.

10. An Agrocybe cylindracea fermented kudzuvine root and coix seeds beverage prepared according to the method of claim 1.
